(19) United States
(12) Patent Application Publication
(10) Pub. No.: US 2003/0138975 A1
(43) Pub. Date: Jul. 24, 2003

(54) Diagnostic signal amplification with proteinoid microspheres

(75) Inventor: Stephen Quirk, Alpharetta, GA (US)

Correspondence Address: SCHWEGMAN, LUNDBERG, WOESSNER & KLUTH, P.A., P.O. BOX 2938, MINNEAPOLIS, MN, 55402, US

(73) Assignee: Kimberly-Clark Worldwide, Inc.

(21) Appl. No.: 10/027,201

(22) Filed: Dec. 20, 2001

(51) Int. Cl. G01N 33/551

(52) U.S. Cl. 436/524

(57) ABSTRACT

The present invention provides modified proteinoid microspheres that can be labeled and/or attached to selective binding moieties. The selective binding moieties can bind to selected targets either in vitro or in vivo. Examples of selective binding moieties include antibodies, ligands, receptors, and the like. Such modified proteinoid microspheres are useful for detecting target antigens, polypeptides, cell types and tissue types. The modified proteinoid microspheres of the invention can also be used for diagnostic imaging.

### FIELD OF THE INVENTION

The invention relates to detection assays and diagnostic imaging using proteinoid microspheres that can amplify the signal from a linked label or a diagnostic imaging agent. The labeled proteinoid microspheres and imaging agents can also include a binding moiety that can interact with and bind to a target, such as an antigen or a specific cell or tissue type so that the antigen, cell or tissue can be detected and/or imaged.

### BACKGROUND OF THE INVENTION

Many available diagnostic tests are antibody based, and can be used to detect either disease-causing agents or biological products produced by the patient in response to the agent. In non-clinical settings antibodies, are often used to detect or isolate natural antigens, for example, enzymes, proteins and peptides that are of interest to researchers.

There are currently three types of antibodies that are used for recognizing antigens: polyclonal antibodies that recognize multiple epitopes, monoclonal antibodies that recognize single epitopes, and molecularly engineered or recombinant phage-displayed and selected antibodies that recognize single epitopes. Each of these antibody types is capable of detecting or identifying an antigen, but the sensitivity of each is limited by the particular immunoassay detection system with which it is used.

Immunoassays, such as enzyme-linked immunosorbent assay (ELISA), enzyme immunoassay (EIA), and radioimmunoassay (RIA), are well known for the detection of antigens. The basic principle of these types of assays is that an enzyme-, chromogen-, fluorophore-, or radionucleotide-conjugated antibody permits antigen detection upon antibody binding. However, it is difficult to detect a signal from a single enzyme, chromogen, fluorophore or radionuclide. In order to reliably detect an antigen with an antibody, significant numbers of antibodies must be bound to a correspondingly large number of antigen epitopes. However, use of a polyclonal antibody that recognizes many epitopes can undermine the specificity of the immunoassay. Moreover, while detection of a single epitope is possible when there is a large concentration of antigen, the sensitivity of a single-epitope immunoassay can be very poor when small amounts of antigen are present. Attaching more enzyme, chromogen, fluorophore or radionuclide to the antibody may help in certain instances. However, addition of too many enzymes, chromogens, fluorophores or radionuclides can diminish the binding affinity or other function of an antibody.

Thus, there is a need for a system that rapidly, reliably, and automatically detects antigens and ligands, especially when they are present in very small quantities.

### SUMMARY OF THE INVENTION

The invention provides a labeled proteinoid microsphere that can be used for a variety of purposes including signal amplification or diagnostic imaging. Such labeled proteinoid microspheres can include a mixture of amino acids that are condensed and a label comprising a fluorophore, a chemiluminescent molecule, a radioisotope, a paramagnetic ion, a metal, or an enzyme. The label can be any such label available to one of skill in the art. For example, the label can be barium sulfate, iocetamic acid, iopanoic acid, ipodate calcium, diatrizoate sodium, diatrizoate meglumine, metrizamide, tyropanoate sodium, fluorine-18, carbon-11, iodine-123, technitium-99m, iodine-131, indium-111, fluorine, gadolinium, fluorescein, isothiocyalate, rhodamine, pacific blue, phycoerythrin, phycocyanin, allophycocyanin, ophthaldehyde, fluorescamine, luminal, isoluminal, luciferin, luciferase or aequorin.

In one embodiment, the proteinoid microsphere is formed by thermal condensation of a mixture of amino acids. In another embodiment, the proteinoid microsphere is formed by condensation of a mixture of amino acids in the presence of a crosslinking agent. The crosslinking agent can be any crosslinking agent, for example, carbodiimide, glutaraldehyde, N-(m-maleimidobenzoyloxy)-succinimide, a bifunctional sulfhydral reagent.

The invention also provides a labeled proteinoid microsphere that is capable of binding to a specific target. Such a proteinoid microsphere comprises a proteinoid microsphere linked to a label and a selective binding moiety that can bind to a specific target. The selective binding moiety can be any molecule available to one of skill in the art that is capable of binding to a specific target. Examples include an antibody, a ligand, a receptor, a peptide, a peptidyl analogue or a polypeptide.

The label can be any label available to one of skill in the art, for example, a fluorophore, a chemiluminescent molecule, a radioisotope, a paramagnetic ion, a metal, or an enzyme. Other examples of labels include barium sulfate, iocetamic acid, iopanoic acid, ipodate calcium, diatrizoate sodium, diatrizoate meglumine, metrizamide, tyropanoate sodium, fluorine-18, carbon-11, iodine-123, technitium-99m, iodine-131, indium-111, fluorine, gadolinium, fluorescein, isothiocyalate, rhodamine, pacific blue, phycoerythrin, phycocyanin, allophycocyanin, ophthaldehyde, fluorescamine, luminal, isoluminal, luciferin, luciferase or aequorin.

In one embodiment, the proteinoid microsphere with the label and the selective binding moiety is formed by thermal condensation of a mixture of amino acids. In another embodiment, the proteinoid microsphere is formed by condensation of a mixture of amino acids in the presence of a crosslinking agent. The crosslinking agent can be any crosslinking agent, for example, carbodiimide, glutaraldehyde, N-(m-maleimidobenzoyloxy)-succinimide, a bifunctional sulfhydral reagent.

The invention also provides a labeled proteinoid microsphere that is capable of binding to a specific target comprising a proteinoid microsphere linked to a label and an antibody that can bind to a specific target. In one embodiment, such a labeled proteinoid microsphere comprises a thermally-condensed mixture of amino acids comprising an acidic amino acid and a basic amino acid. The label can be any label available to one of skill in the art, for example, a fluorophore, a chemiluminescent molecule, a radioisotope, a paramagnetic ion, a metal, or an enzyme. The antibody is also any antibody available to one of skill in the art that can selectively bind to a target chosen by one of skill in the art. The acidic amino acid can be, for example, aspartic acid or glutamic acid or a mixture of both. The basic amino acid can be, for example, arginine or lysine, or a mixture of both. The mixture of amino acids can further comprise cysteine.

Such labeled proteinoid microspheres with selective binding moieties can be used assays and testing procedures. For example, labeled proteinoid microspheres with selective binding moieties can be used in immunoassays such as radioimmunoassays, ELISAs, immunofluorescence assays or a sandwich assays. In another embodiment, such labeled proteinoid microspheres are used for diagnostic imaging or signal amplification.

Signal amplification provided by the present labeled proteinoid microspheres that are linked to antibody selective binding moieties is at least about thirty-fold relative to an antibody preparation linked to the same label.

### DESCRIPTION OF THE FIGURES

FIG. 1 provides a schematic diagram of a proteinoid microsphere (O), linked to an antibody (Y), and a fluorophore (*). The actual number of linked antibodies and fluorophores can vary. Generally, there is greater than one antibody and one fluorophore is present per proteinoid microsphere.

FIG. 2 provides a comparison of steady state fluorescence emission intensity spectra for a complex of the PM4 proteinoid microsphere preparation linked to Pacific Blue and to a monoclonal antibody (highest curve at 450 nm), an equal amount of Pacific Blue modified mAb (middle curve at 450 nm), and a mixture of unreacted mAb and PM4 (lowest curve at 450 nm).

FIG. 3 provides results of an ELISA titration assay. Relative fluorescence intensity is plotted as a function of the log of the dilution of either the PM4-flourophore-mAb complex (filled circles) or an anti-human serum albumin monoclonal antibody (open circles).

FIG. 4 provides an ITC analysis of the interaction of the PM4 complex with human serum albumin. Each peak shows the heat produced by the injection and subsequent binding reaction. Bottom panel: Binding isotherm produced by integrating each injection peak with respect to time.

FIG. 5 provides an SPR binding analysis of the PM4 complex binding to a surface of immobilized HSA-anti HSA pAb.

### DETAILED DESCRIPTION OF THE INVENTION

The invention provides proteinoid microspheres that have both binding moieties and labels for use in diagnostic tests, diagnostic imaging and for detection of any antigen, ligand, receptor, or other molecule recognized by the binding moiety.

Proteinoid Microspheres

Thermally condensed amino acids can form spherical particles (Fox 1964). Proteinoid microspheres made in this manner can vary in size from 1 to 10 microns and many of them are hollow. Once formed, thermal proteinoid microspheres are stable (Muller-Herold and Nickel, 1994; Syren, et al., 1985).

Proteinoid microspheres are relatively easy to synthesize and characterize (Fox and Nakashima, 1966; Phillips and Melius, 1974; Luque-Romero et al., 1986; Kokufuta et al., 1983). For example, U.S. Pat. No. 4,925,673 (the '673 patent) describes proteinoid microspheres as well as methods for their preparation and use. Proteinoid microspheres can, for example, be formed by the thermal condensation of amino acids. Thermal condensation joins carboxyl and amino groups that are part of an amino acid side chain or that are in the α position. A generalized scheme for a proteinoid thermal condensation reaction is illustrated below.

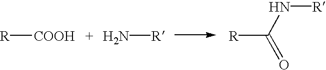

The carboxylate can be donated from either an amino acid side chain (Asp or Glu) or from the α carboxylate. Similarly, the free amino group can be donated from either an amino acid side chain (Arg, Lys, Asn, or Gln) or from the α amino group. It is relatively straightforward to produce proteinoids that are either acidic or basic via this simple thermal condensation reaction.

While most applications do not require it, a crosslinking reagent can be used to stabilize the structure of the proteinoid microspheres. The incorporation of such a crosslinking agent has no deleterious effects on the formation of the proteinoid microspheres or on their subsequent functioning as labeling and diagnostic imaging agents. For many applications, no crosslinking agent is needed because the proteinoid microspheres of the invention are sufficiently stable, durable and soluble without crosslinking.

By way of example, proteinoid microspheres can be made by mixing dry amino acids with or without a cross-linking reagent, for example, by grinding in a mortar and pestle until completely mixed. Mixing time can vary as needed. Approximate times for mixing are five minutes to one hour. In one embodiment, mixing is done for about thirty minutes. The resulting powder can be further mixed via sonication, for example, using a water-sonicating bath. Sonication can be done for about ten minutes to about four hours. In one embodiment, sonication was for about two hours.

Crosslinking agents useful for formation of the proteinoid microspheres of the invention include any crosslinking reagent known to one of skill in the art. Examples of crosslinking agents can be found in the Pierce Chemical Co. 2000-2001 catalog, and include such agents as carbodiimide, glutaraldehyde, N-(m-maleimidobenzoyloxy)-succinimide, and the like.

A crosslinking reagent can be incorporated during or after formation of the proteinoid microspheres. The crosslinking reagent is incorporated via condensation between the reactive groups of the crosslinking reagent and the free amino or carboxylate groups of the amino acids. In one embodiment, the reactive group on the crosslinking agent is a carboxylate that condenses with free amino groups of the amino acids used to form the proteinoid microsphere. In another embodiment, a reactive group is chosen that does not react with the amino or carboxylate groups of the amino acids. Instead, the reactive group is chosen to react with a selected amino acid side chain. Then, the amount of cross-linking can be controlled by varying the amount of the selected amino acid side chain in the reaction mixture.

The amount of crosslinking can also be controlled by varying the amount of the crosslinking reagent. Hence, the amount of crosslinking reagent used can vary. For example, the crosslinking reagent can form about 0.01% to about 20% of the proteinoid microsphere composition. Desirable proteinoid microsphere compositions contain about 0.1% to about 15% crosslinking reagent. More desirable proteinoid microsphere compositions contain about 1% to about 10% crosslinking reagent.

Some branching between crosslinked and thermally condensed amino acids is desirable to facilitate formation of spheroidal proteinoids. Hence, the type and amount of crosslinking reagent selected should permit branching. Care should be taken to ensure that excessive crosslinker is not used so that branching is eliminated or excessively inhibited. The type of amino acid utilized in the proteinoid microspheres can also influence the amount of branching. For example, when the reactive group on the crosslinking reagent reacts with carboxylate and/or amino groups, amino acids with carboxylate and/or amino group side chains can be used to encourage branching.

The amino acid-crosslinker powder mixture is heated at a first temperature for several hours. The first temperature can vary, for example from about 150° C. to about 220° C. In one embodiment, the first temperature was about 190° C. Heating times at this first temperature can also vary, for example, from six to twelve hours. In one embodiment, the mixture was heated at about 190° C. for about nine hours. After heating at the first temperature, the temperature was raised to a second, higher temperature for several more hours. The second temperature can also vary, for example, from temperature about 200° C. to about 220° C. In one embodiment, the second temperature was about 220° C. The time for heating at this second temperature can vary, for example, from about one hour to about six hours. In one embodiment, the mixture was heated at a second temperature of about 220° C. for about three hours. It is desirable to maintain a blanket of dry nitrogen gas over the amino acid mixture at all times. Over the course of the reaction, the mixture may change from an off white powder to a yellowish-brown semi-solid.

After heating, the reaction mixture is cooled to room temperature, and the solid material is resuspended in an excess of water. This process can be aided by sonication and stirring. Insoluble matter can be removed from the mixture by centrifugation, for example, at 3,000×g for 10 minutes at 25° C. The supernatant from such a centrifugation is collected. The proteinoid materials can be further purified by dialysis against water using, for example, a 3500 MWCO dialysis membrane. The proteinoid materials can be dried for easy storage, for example by lyophilization.

Amino Acids

Amino acids that can be used in the proteinoid microspheres of the invention can be genetically encoded L-amino acids, naturally occurring non-genetically encoded L-amino acids, synthetic L-amino acids or D-enantiomers of any of the above. The amino acid notations used herein for the twenty genetically encoded L-amino acids and common non-encoded amino acids are conventional and are as shown in Table 1.

TABLE 1

| | | Common |
|---|---|---|
| Amino Acid | One-Letter Symbol | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | bAla |
| 2,3-Diaminopropionic acid | | Dpr |
| α-Aminoisobutyric acid | | Aib |
| N-Methylglycine (sarcosine) | | MeGly |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydro- | | Tic |
| isoquinoline-3-carboxylic | | |
| acid | | |
| β-2-thienylalanine | | Thi |
| Methionine sulfoxide | | MSO |
| Homoarginine | | hArg |
| N-acetyl lysine | | AcLys |
| 2,4-Diamino butyric acid | | Dbu |
| ρ-Aminophenylalanine | | Phe(pNH₂) |
| N-methylvaline | | MeVal |
| Homocysteine | | hCys |
| Homoserine | | hSer |
| ε-Amino hexanoic acid | | Aha |
| δ-Amino valeric acid | | Ava |
| 2,3-Diaminobutyric acid | | Dab |

Surprisingly, so long as free amino and carboxylate groups from the amino acids are available for condensation, the amino acid composition plays a minimal role in proteinoid microsphere structure and function. Hence, any of the above amino acids, including both D and L enantiomers of these amino acids can be incorporated into the proteinoid microspheres. Even non-amino acids can be incorporated into or onto proteinoid microspheres, so long as amino and/or carboxylate groups are available for condensation. Amino acid mixtures for forming proteinoid microspheres can therefore be as complex or as simple as desired.

Such flexibility in composition permits incorporation of naturally occurring, non-genetically encoded amino acids that may have useful reactive groups for attachment of labels and selective binding moieties. Additional or selected functional groups for linking labels and binding moieties to the proteinoid microspheres can be built into a proteinoid microsphere by choosing amino acids with desirable side chain moieties or other properties. For example, amino acids as tyrosine can be added to provide hydroxyl groups, cysteine can be added to provide thiol groups, phenylalanine or other hydrophobic amino acids can be added to alter the solubility and proteinoid microsphere binding character and tryptophan can be added to form an intrinsic fluorescence probe.

Therefore, one useful property of the present proteinoid microspheres is that there is a wide degree of latitude to their construction and composition. This latitude means that proteinoid microspheres can be individually tailored to provide desired linking groups, solubilities and to perform as desired in any environment.

Selective Binding Moieties

A Selective Binding Moiety including any antibody, ligand, receptor, nucleic acid, nucleic acid aptamer, peptide nucleic acid, peptide, peptidyl analogue or polypeptide that can associate with or bind to a specific target that is of interest to one of skill in the art. Any target of interest to one of skill in the art is contemplated. Such a target can, for example, be an antigen, a cell or a tissue that is diagnostic of a disease state. Binding by the Selective Binding Moiety to the target should be sufficiently stable to permit detection or imaging of the target and sufficiently selective to permit discrimination and identification of the target in an in vitro mixture or in a complex in vivo environment.

One or more Selective Binding Moieties are linked to the proteinoid microspheres of the invention. Linkage between a proteinoid microsphere and a Selective Binding Moiety can be via a covalent bond, or via any other stable interaction.

A Selective Binding Moiety can be an antibody, a ligand for a receptor, a receptor for a ligand, a membrane-associated domain for any cell membrane protein known to one of skill in the art, an inhibitor for a membrane-associated protein, a component that binds to, or is generally associated with, tumor cells, a component that binds to, or is generally associated with, tumor vasculature, a component of the tumor extracellular matrix or stroma, a cell found within the tumor vasculature or any peptide or polypeptide that preferentially interacts with a cellular membrane.

Selective Binding Moieties can be made to bind to any relatively specific marker on the tumor cell. Many so-called “tumor antigens” have been described, any one which could be employed as a target to which the Selective Binding Moiety may bind. In many cases, antibodies are available that can act as Selective Binding Moieties of these targets.

Antibodies as Selective Binding Moieties

The invention provides antibody preparations directed against specific antigenic targets of interest to one of skill in the art. Such antibodies can be Selective Binding Moieties that can be linked to the proteinoid microspheres of the invention to permit detection and diagnostic imaging of the target.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen-binding region referred to as a variable region and a non-varying region referred to as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely a adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector function, such as participation of the antibody in antibody-dependent cellular toxicity.

An antibody that is contemplated for use in the present invention thus can be in any of a variety of forms, including, but not limited to a whole immunoglobulin, an antibody fragment such as Fv, Fab, and similar fragments, a single chain antibody which includes the variable domain complementarity determining regions (CDR), and so forth, all of which fall under the broad term “antibody,” as used herein. The present invention contemplates the use of any specificity of an antibody, polyclonal or monoclonal.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments retain some ability to selectively bind with its antigen or receptor and are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H} -V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad Sci. USA 90: 6444-6448 (1993).

The preparation of polyclonal antibodies is well-known to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

The preparation of monoclonal antibodies likewise is conventional. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of monoclonal antibodies are well known to those skilled in the art. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol Biol. 222: 581-597 (1991). Another method involves humanizing a monoclonal antibody by recombinant means to generate antibodies containing human specific and recognizable sequences. See, for review, Holmes, et al., J. Immunol., 158:2192-2201 (1997) and Vaswani, et al., Annals Allergy, Asthma & Immunol., 81:105-115 (1998).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad Sci. 81, 6851-6855 (1984).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces twp monovalent Fab′ fragments and an Fc fragment directly. These methods are described, for example, in U.S. Pat. Nos. 4,036,945 and 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or subtantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

In vitro and in vivo Detection Methods

The labeled proteinoid microspheres of the invention can amplify a signal from any label or imaging agent. While not wishing to be limited to a specific mechanism, it appears that more label may be concentrated or bound to target using the present labeled proteinoid microspheres with attached selective binding moieties than when using just a labeled selective binding moiety. This may be because several labels or imaging agents and several selective binding moieties can be linked to the same proteinoid microsphere, thereby concentrating the label on the target once the selective binding moiety is bound to target. The degree of signal amplification can vary and may be adjusted by one of skill in the art to provide the level of signal or degree of detection needed for a particular assay or detection device.

In a desirable embodiment, the labeled, proteinoid microspheres of the invention that have attached selective binding moieties may amplify a signal by up to about 50-fold, relative to the same selective binding moiety that is labeled with the same label but is not attached to a proteinoid microsphere. In a more desirable embodiment, the labeled proteinoid microspheres of the invention that have attached selective binding moieties may amplify a signal by up to about 75-fold. In an even more desirable embodiment, the labeled proteinoid microspheres of the invention that have attached selective binding moieties may amplify a signal by up to about 100-fold, relative to the same selective binding moiety that is labeled with the same label but is not attached to a proteinoid microsphere. In one embodiment, the labeled proteinoid microspheres of the invention that have attached selective binding moieties can amplify a signal by at least about 30-fold. For example, fluorescently-labeled proteinoid microspheres of the invention to which antibodies directed against human serum albumin (HSA) were attached, amplified the signal about 30-fold relative to fluorophore-labeled anti-HSA antibodies.

Any convenient test sample may be screened or tested. For example, the sample may be a clinical sample such as blood, serum, sweat, urine, semen, vaginal secretions, menses, cerebrospinal fluid and so forth. The test sample may also be a tissue or cell sample. The test sample may be an environmental sample such as soil, water, food and so forth.

The labeled proteinoid microspheres of the invention can be used in any testing or diagnostic procedure. The testing or diagnostic procedure may be qualitative or quantitative. For example, the labeled proteinoid microspheres with antibodies as Selective Binding Moieties can be used in any immunoassay technique. Immunoassays contemplated by the invention include radioimmunoassays, ELISAs, immunofluorescence assays, sandwich assays and the like. Such procedures are described, for example, in O'Sullivan, Annals Clin. Biochem., 16:221-240 (1976); McLaren, Med. Lab. Sci., 38:245-51 (1981); Ollerich, J. Clin. Chem. Clin. Biochem., 22:895-904 (1984); Ngo and Lenhoff, Mol. Cell. Biochem., 44:3-12 (1982).

A labeled proteinoid microsphere with an antibody as a Selective Binding Moiety can also be used in vivo as an imaging agent. After administration to a patient, the location of the imaging agent may be tracked by use of a detector sensitive to the label.

A variety of imaging techniques have been used to diagnose diseases. One early method was X-ray imaging. The images produced using X-rays reflect the different densities of structures and tissue in the body of the patient. X-ray imaging of soft tissues was problematic until the development of constrast agents that can increase the density of tissues of interest relative to surrounding tissues.

The present invention therefore provides proteinoid microspheres comprising desirably linked or attached contrast agents. Contrast agents include, for example, barium and iodinated compounds. These proteinoid microsphere-contrast agents may be used for X-ray studies of the gastrointestinal region, including the esophagus, stomach, intestines and rectum. Contrast agents may also be used for computed tomography (CT) and computer assisted tomography (CAT) studies to improve visualization of tissue of interest, for example, the gastrointestinal tract.

Magnetic resonance imaging (MRI) is an imaging technique that, unlike X-rays, does not involve ionizing radiation. MRI may be used for producing cross-sectional images of the body in a variety of scanning planes such as, for example, axial, coronal, sagittal or orthogonal. MRI employs a magnetic field, radio frequency energy and magnetic field gradients to make images of the body. The contrast or signal intensity differences between tissues mainly reflect the T1 (longitudinal) and T2 (transverse) relaxation values and the proton density, which generally corresponds to the free water content, of the tissues. To change the signal intensity in a region of a patient by the use of a contrast medium, several possible approaches are available. For example, a contrast medium may be designed to change the T1, the T2 or the proton density.

MRI also requires the use of contrast agents. Paramagnetic contrast agents have frequently been used for MRI. Paramagnetic contrast agents involve materials that contain unpaired electrons. The unpaired electrons act as small magnets within the main magnetic field to increase the rate of longitudinal (T1) and transverse (T2) relaxation. Paramagnetic contrast agents typically comprise metal ions, for example, transition metal ions, which provide a source of unpaired electrons. However, these metal ions are also generally highly toxic. In an effort to decrease toxicity, the metal ions are typically chelated with ligands.

Paramagnetic contrast agents that can be used with the present invention for MRI include paramagnetic agents such as metal oxides, most notably iron oxides and nitroxides as well as gadolinium ions that are often complexed as Gd-DTPA. Numerous other examples of elements useful in magnetic resonance imaging are available and may be used as labels within the context of the present invention. For discussions on in vivo nuclear magnetic resonance imaging, see, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415; Kornmesser et al., Magnetic Resonance Imaging, 6:124 (1988).

Ultrasound is another valuable diagnostic imaging technique for studying various areas of the body, including, for example, the vasculature, such as tissue microvasculature. Ultrasound provides certain advantages over other diagnostic techniques. For example, diagnostic techniques involving nuclear medicine and X-rays generally involve exposure of the patient to ionizing electron radiation. Such radiation can cause damage to subcellular material, including deoxyribonucleic acid (DNA), ribonucleic acid (RNA) and proteins. Ultrasound does not involve such potentially damaging radiation. In addition, ultrasound is relatively inexpensive relative to other diagnostic techniques, including CT and MRI, which require elaborate and expensive equipment.

Ultrasound involves the exposure of a patient to sound waves. Generally, the sound waves dissipate due to absorption by body tissue, penetrate through the tissue or reflect off of the tissue. The reflection of sound waves from tissue, generally referred to as backscatter or reflectivity, forms the basis for developing an ultrasound image. Sound waves reflect differentially from different body tissues. This differential reflection is due to various factors, including the constituents and the density of the particular tissue being observed. Ultrasound machines detect such differentially reflected waves, generally with a transducer that can detect sound waves having a frequency of one megahertz (MHZ) to ten MHZ. The detected waves can be integrated into an image that is quantified and the quantified waves are converted into an image of the tissue being studied.

Like the diagnostic techniques discussed above, ultrasound generally involves the use of contrast agents. Exemplary contrast agents for ultrasound techniques include, for example, suspensions of solid particles, emulsified liquid droplets, and gas-filled bubbles. See, e.g., Hilmann et al., U.S. Pat. No. 4,466,442, and published Patent Applications WO 92/17212, WO 92/21382 and EP-A-0 324 938 (disclosing stabilized microbubble-type ultrasonic imaging agents produced from heat-denaturable biocompatible protein, for example, albumin, hemoglobin, and collagen). The reflection of sound from a liquid-gas interface is extremely efficient.

The present invention therefore contemplates use of proteinoid microspheres for use a ultrasound contrast agents. Such proteinoid microspheres can be filled with an appropriate liquid or solid material, as desired by one of skill in the art. As known to the skilled artisan, the signal that is reflected off of an ultrasound imaging agent is a function of the radius (r⁶) of the bubble (Rayleigh Scatterer). Thus, in the frequency range of diagnostic ultrasound, an imaging agent having a diameter of 4 micrometer (μm) possesses about 64 times the scattering ability of a bubble having a diameter of 2 μm. Thus, generally speaking, the larger the proteinoid microsphere imaging agent, the greater the reflected signal.

However, the size of a diagnostic imaging agent is necessarily limited by the size of the blood vessels through which it must pass. Generally, diagnostic imaging agents having a diameter of greater than 10 μm are not desirable because they may occlude smaller microvessels. Accordingly, it is generally desirable for greater than about 99% of the proteinoid microsphere diagnostic imaging agents of the invention to have a diameter of less than 10 μm.

For proteinoid microsphere imaging agents, the diameter is also important, and should be greater than 1 μm, with greater than 2 μm being preferred.

Labeling Proteinoid Microspheres

The proteinoid microspheres of the invention can be labeled for use in a wide variety of detection and diagnostic imaging procedures. Many labels and procedures are available in the art for labeling and are applicable to the proteinoid microspheres of the invention. For example, the proteinoid microspheres of the invention can be labeled with a radioactive label, a diagnostic imaging agent, an enzyme or a fluorescent dye. When the proteinoid microsphere is fully formed, a label can be attached to the exterior or loaded into the interior of the proteinoid microsphere.

To infuse a label into a preparation of proteinoid microspheres, the proteinoid microspheres are suspended in an aqueous solution, for example, a buffer or a buffered saline solution. The label or diagnostic imaging agent is then added to the suspension at a concentration that will yield a proteinoid microsphere preparation that contains a detectable amount of the label or diagnostic imaging agent within the microspheres. The suspension is then heated for a time and to a temperature sufficient for the label or diagnostic imaging agent to enter the proteinoid as the microsphere forms. For example, in one embodiment, the proteinoid microspheres were heated to about 50° C. for about 15 minutes. The suspension is allowed to slowly cool to an temperature convenient for handling, for example, room temperature. During the cooling process microspheres form and precipitate from solution. The microspheres can be dialyzed versus or filtered to remove unencapsulated materials. If desired, the microspheres can be dried prior to use. Such a loaded proteinoid microsphere complex is stable. A molecule, label or diagnostic imaging agent trapped in the proteinoid microsphere interior will remain inside.

A detectable label can also be attached to the exterior of a proteinoid microsphere preparation. The label may be a radiolabel, fluorophore, or enzyme that is directly or indirectly conjugated to a reactive group on the proteinoid microsphere. Examples of labels include, but are not limited to, barium sulfate, iocetamic acid, iopanoic acid, ipodate calcium, diatrizoate sodium, diatrizoate meglumine, metrizamide, tyropanoate sodium, fluorine-18, carbon-14, iodine-125, technitium-99m, iodine-131, indium-111, fluorine, gadolinium, fluorescein, isothiocyalate, rhodamine, pacific blue, phycoerythrin, phycocyanin, allophycocyanin, ophthaldehyde, fluorescamine, luminal, isoluminal, luciferin, luciferase or aequorin. Those of ordinary skill in the art will know of other suitable labels that may be employed in accordance with the present invention.

In general, fluorescent or phosphorescent dyes are desirable and suggested labels for the present proteinoid microspheres. Examples of fluoresecent dyes include Pacific Blue, fluorescein, rhodamine, Texas Red.RTM, cyanine, merocyanine, phycoerythrin and styryl fluorescent dyes. One of skill in the art can link such fluorescent dyes onto the proteinoid microspheres of the invention, for example, via the amino, carboxyl or side chain moieties of the amino acids used to form the proteinod microspheres. Similarly, detection procedures involving a luminescence or light absorption by fluorophore labels are generally available. For example, the detecting step can be an optical detecting step wherein the liquid is illuminated with light of first defined wavelengths. Light at a second defined wavelength(s) that is fluoresced or phosphoresced by the labeled microsphere may be then detected. The detection also can be by optical light absorption. For example, the detecting step can comprise passing light of first defined wavelengths through the liquid and then ascertaining the wavelength of the light that is transmitted by the liquid.

While fluorescent labels are suggested, other types of labels can be used with the proteinoid microspheres of the invention. For example, radiometals can be bound to proteinoid microspheres via sulfhydryl groups. Generally, such radiometals are metal ions such as the ions of Tc-99m, Re-186, Re-188, Cu-64, Cu-67, Hg-195, Hg-197, Hg-203, Pb-203, Pb/Bi-212, Zn-72, Au-198, Au-199, Cd-115, Cd-115m, Sn-117, Sn-125, and the like. Radiometals having gamma emission energies in the range of about 50-500 KeV are useful for scintigraphy. Positron emitters can also be used for imaging applications. Beta and alpha emitters are useful for therapy. Preferably, the radiolabeling yield is greater than 80%, and, more preferably, is greater than 90%.

In one embodiment, the label is a pertechnate, rhennate, or other radioisotopic agent of similar chemistry. In general, the pertechnetate or rhennate will be reduced so that it will react with the free thiol groups of the proteinoid microspheres. Suitable reducing agents include but are not limited to sources of stannous ion, such as stannous chloride and stannous tartrate; stannous tartrate is preferred because the tartrate anion stabilizes the Sn—Tc complex. Other reducing agents known in the art include 2-mercaptoethanol, 1,4-dithiothreitol, 2,3-dihydroxybutane-1. 4-dithiol, 2-aminoethanethiol HCl, 2-mercaptoethylamine, thioglycolate, cyanide and cysteine. The amount of the reducing agent, and the incubation time, are adjusted in the light of the reducing agent employed.

Technetium-99m is a desirable radiolabel for scintigraphy because of its ready availability and ease of preparation from commercial pertechnetate generators. Technetium labeling of the sulfhydryl-containing proteinoid microspheres is generally effected by conventional methods. Pertechnetate is obtained from a commercially available generator, most commonly in the form of NaTcO₄, normally in saline solution. Other forms of pertechnetate may be used, with appropriate modification of the procedure, as would be suggested by the supplier of a new form of generator or as would be apparent to a person of skill in the art.

Pertechnetate is generally used at an activity of about 0.2-10 mCi/ml in saline, e.g., 0.9% (“physiological”) saline, buffered at a pH of about 3-7, preferably 3.5-5.5, more preferably about 4.5-5.0. Suitable buffers include, e.g., acetate, tartrate, phthalate, citrate, phosphate and so forth

Rhenium is found just below technetium in the periodic table and has the same outer shell electronic configuration. Rhenium and its compounds are expected to have very similar chemical properties to technetium and its analogous compounds. In fact, rhenium compounds behave similarly to technetium compounds insofar as reduction and chelation are concerned but their greater susceptibility to oxidation requires greater care in handling.

The radioisotope Re-186 is attractive for both imaging and therapy. It has a half-life of about 3.7 days, a high LET beta emission (1.07 MeV) and a convenient gamma emission energy (0.137 MeV). Rhenium may be produced from perrhenate, and the reduced rhenium ions can bind non-specifically to proteinoid microspheres. Accordingly, a method for Re-186 labeling of proteinoid microspheres, wherein the reduced perrhenate is bound to sulfhydryl groups, would be advantageous. Re-188 is a generator-produced beta and gamma emitter with a half-life of about 17 hours and could be useful for imaging and therapy.

Rhenium labeling is performed in substantially the same manner as technetium labeling, with special care being taken to ensure the absence of air or other source of oxygen from the system. Re-186 is produced in the form of sodium perrhenate by use of a generator analogous to currently available technetium generators.

By “reduced pertechnetate” or “reduced perrhenate” is meant the species of technetium or rhenium ion formed by chemical reduction of pertechnetate or perrhenate and chelated by the thiol group(s). It is generally thought that reduced pertechnetate is in the form of Tc(III) and/or Tc(IV) and/or Tc(V) in such chelates and that reduced perrhenate is in the form of Re(III) and/or Re(IV) and/or Re(V), but higher or lower oxidation states and/or multiple oxidation states cannot be excluded and are within the scope of the invention.

Copper will normally be in the form of Cu(II), although Cu(I) and/or Cu(II) are not excluded. Mercury will normally be in the form of Hg(I) and/or Hg(II). Lead/bismuth will normally be in the form of Pb(II) or Pb(IV).

Reduction is effected by any of a variety of conventional reducing agents, preferably stannous ion generally in aqueous solution. Other suitable reducing agents include, e.g., dithionite, borohydride, ferrous ion, formadine sulfonic acid, and so forth. It will be appreciated that stannous ion can be generated in situ from tin metal, e.g., foil, granules, powder, turnings and the like, by contact with aqueous acid, e.g., HCl.

Copper ions are also tightly chelated by sulfur chelators. Cu-67 is another attractive radionuclide for imaging and therapy. It has a half-life of about 2.6 days, and is a beta (0.570 MeV) and gamma emitter (0.185 MeV), although the beta energy is relatively low. Cu-67 is relatively expensive and not readily available at present, although such conditions can change as demand evelops. It has the advantage that it forms tight chelates with thiols. The labeling is simple and rapid, and requires no reducing agent for the radiometal.

Copper labeling will be effected by reaction of a thiol-containing proteinoid microspheres with a solution of copper ions, normally Cu(II) ions, in the form of a convenient salt, e.g., chloride, citrate, tartrate or the like, either as available or by mixing of e.g., the chloride with, e.g., sodium, potassium or ammonium citrate, tartrate or the like. Cu-67 is currently available as CuCl.sub.2 from Oak Ridge National Laboratories, Tennessee, or from Los Alamos National Laboratories, N. Mex. Zinc, silver, gold and cadmium isotopes would chelate SH groups in a manner similar to copper.

Other radionuclides with similar chelation behavior to copper, e.g., mercury and lead, also could be bound to thiol-containing compounds according to the method of the invention. Hg-197 has a half-life of about 1.5 days, and emits gamma radiation in an energy range of 78-268 KeV, and Pb-203 is a strong gamma-emitter at about 275 KeV, with a half-life of about 51 hr, making them suitable for gamma scintigraphy. Bi-212 is an alpha emitter with a half-life of about 1 hr and an energy of 6.09 MeV, making it of considerable interest for in vivo therapy. It is produced in situ from a Pb-212 precursor with emission of gamma radiation of 239 KeV, with a half-life of about 10.6 hr. Thus, antibody conjugates for Bi-212 therapy will be Pb-212 labeled conjugates, and the short-hand notation lead/bismuth or Pb/Bi is used herein to indicate this. Chelation to the antibody protein is effected analogously to Cu-67 labeling.

Mercury radioisotopes are normally available as HgCl₂ or as Hg(NO₃)₂, e.g., from Oak Ridge National Laboratories. Lead/bismuth radioisotopes are normally available from Argonne National Laboratories in the form of supported radon generator. Stable isotopes may also be conjugated to proteins for therapeutic (e.g., Au for arthritis) or diagnostic (e.g., colloidal Au compounds for electron microscopy) purposes.

Chemical analysis methods available to one of skill in the art can be used to detect attachment of the label to the proteinoid microspheres of the invention. Chemical analysis methods can include infrared spectrometry, NMR spectrometry, absorption spectrometry, fluorescence spectrometry, mass spectrometry, radioactive detection, scintillation counter detection and chromatographic methods.

The invention will be further described by reference to the following detailed examples, which are given for illustration of the invention, and are not intended to be limiting thereof.

### EXAMPLE 1

### Materials and Methods

Materials

L-amino acids and buffer reagents were obtained from Sigma Chemical Co. (St. Louis, Mo). Pacific Blue fluorophore was acquired from Molecular Probes, Inc.

Proteinoid Formation

The PM4 proteinoid preparation consisted of 1.0 g of aspartic acid, 1.0 g of glutamic acid, 1.0 g of asparagine, 1.0 g of arginine, and 1.0 g of serine. Synthesis of proteinoids generally followed the procedures outlined in the scientific literature (e.g.—Fox and Nakashima, 1966; Phillips and Melius, 1974; Luque-Romero et al., 1986). Specifically, dry amino acids were mixed together and ground in a mortar and pestle until completely mixed (approximate time 30 minutes). The resulting powder was further mixed via sonication in a water sonicating bath for two hours. The final powder was transferred into a glass vessel and was immersed into a mineral oil bath.

The mixture was heated in the mineral oil bath at a temperature of 190° C. for nine hours. After nine hours, the temperature was raised to 220° C. for three hours. A blanket of dry nitrogen gas was maintained over the amino acid mixture at all times. Over the course of the reaction, the mixture changed from an off white powder to a yellowish-brown semi-solid. The reaction was cooled to room temperature, and the solid material was resuspended in an excess of water (usually 10 mLs). This process was aided by sonication and stirring. The mixture was centrifuged at 3,000×g for 10 minutes at 25° C. in order to remove insoluble matter. The decanted solution was dialyzed extensively against water (1 L with multiple changes over a 48 hour period at 25° C.) using a 3500 MWCO dialysis membrane. The contents of the dialysis bag were returned to dryness via lyophilization.

Microsphere Formation

Dried proteinoids (200 mg) were resuspended in 20 mL of 10 mM Tris (pH 7.1). The solution was heated to 50° C. for 15 minutes in a beaker containing 100 mL of water. The beaker was removed from the hot plate and was allowed to come to room temperature over an approximate one-hour period. During the cooling process microspheres formed and precipitated from solution. The mixture was either dialyzed versus 1 L of 10 mM Tris (pH 7.1) overnight at room temperature or was filtered through Whatman filter paper, washed with 10 mM Tris (pH 7.1), and collected. The microspheres were dried prior to use.

Chemical Coupling of a Fluorophore

The fluorophore used was Pacific Blue from Molecular Probes, Inc. The structure of Pacific Blue is provided below.

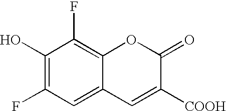

The excitation and emission wavelengths used for the Pacific Blue fluorophore were 405 nm and 450 nm respectively.

PM4 was resuspended in HEPES buffered saline (HBS, 10 mM HEPES, pH 7.4; 150 mM NaCl; 3 mM EDTA) at a final concentration of 20% (v/v). Pacific Blue succinimide ester (5 mg) was dissolved in a small volume of dimethylsulfoxide and added to the vial containing PM4. The reaction was stirred slowly at room temperature for one hour in the dark. The PM4 preparation was dialyzed versus HBS at room temperature for 12 hours.

Antibody Thiolation

An anti human albumin monoclonal antibody (mAb) was diluted into phosphate buffered saline (PBS, 10 mM KPi, pH 7.4; 150 mM NaCl) to a final concentration of 2 mg/mL. A fresh stock of 1.2 mM Sulfo-LC-SPDP (Pierce) was added to the antibody solution to a final concentration of 0.12 mM. The reaction mixture was stirred at room temperature for 60 minutes. The thiolated mAbs were purified via a 5 mL desalting column run in PBS. Fractions containing protein were pooled and concentrated to 10 mg/mL via Centricon (Amicon, Inc.).

Chemical Coupling of an Anti Human Albumin mAb

The PM4-Pacific Blue complex was reacted with 50 mM N-hydroxysuccinimide, 0.2 M N-ethyl-N′-(dimethylaminopropyl)-carbodiimide in HBS at room temperature (with slow mixing) for 30 minutes. 2-(2-pyridinyldithio) ethaneamine (PDEA) was dissolved in 0.1 M Borate buffer (pH 8.5) to a final concentration of 80 mM and added to the PM4 complex (final concentration 40 mM). This mixture was stirred at room temperature for one hour in the dark. Thiolated anti human albumin monoclonal antibody was added to the stirring PM4 mixture. Typically 2 mg of mAb was used per coupling reaction. The mixture was incubated at room temperature for an hour with gentle stirring. Cystamine-HCl was added to the reaction at a final concentration of 40 mM. Incubation continued for an additional thirty minutes. The final PM4-Pacific Blue-mAb complex (hereafter referred to as the PM4 complex) was purified from unreacted species by filtering the mixture through Whatman filter paper, washing with 10 mM Tris (pH 7.1), and collecting the modified PM4 complex.

Fluorescence Assays

All fluorescence experiments were conducted using a Shimadzu RF5301 fluorometer. Samples were slowly stirred to avoid proteinoid microsphere settling for both kinetic and steady state measurements.

ELISA Analysis

One microgram of human serum albumin was adsorbed to the surface of a 96-well microtiter plate. The wells were then blocked with 10% non-fat dry milk in PBS. The blocked wells were washed three times with PBS. The PM4 complex and the anti human mAb were adjusted to the same protein concentration via the Bradford assay (Bradford, 1976) and serial dilutions were prepared in PBS. Aliquots of these dilutions were added into the microtiter wells and were allowed to react at room temperature for one hour. The wells were then washed three times with PBS. For the assay that employed the regular mAb, a goat anti-mouse secondary antibody, conjugated to Pacific Blue (Molecular Probes, Inc.), was utilized for the detection phase of the ELISA. A 1:2000 dilution of the secondary antibody conjugate was added into the microtiter wells and was allowed to incubate at room temperature for one hour. These wells were then washed three times with PBS. Alternatively, the anti human serum albumin monoclonal antibody was labeled directly with Pacific Blue succinimide ester. Fluorophore to mAb ratios were employed in the labeling reaction such that one molecule of Pacific Blue was bound per antibody molecule. The fluorescence emission intensity was determined using a Dynex, Inc. fluorescent microtiter plate reader employing a 410 nm (excitation) and a 460 nm (emission) bandpass filter set. Absolute fluorescence was converted to relative fluorescence by setting the fluorescence value at the highest mAb (or PM4 complex) concentration to 100%.

Isothermal Titration Calorimetry

Isothermal titration calorimetry (ITC) was performed with a VP-ITC instrument from MicroCal, Inc. Titrations were carried out by injecting 5 μL of an anti human serum albumin monoclonal antibody (at concentrations that ranged from 50 to 200 μM) into the 1.4 mL stirred reaction cell. PM4 ranged in concentration from 5 to 10% (v/v) in the cell. Both the mAb and PM4 were in 20 mM sodium cacodylate (pH 5.5), 40 mM NaCl. Titrations were conducted at 20° C. Typical experimental conditions for the titrations were a 10 second injection period followed by a 240 second delay between injections for a total of 40 injections. Blank titrations of antibody into buffer were performed in order to correct for heats of dilution and mixing.

The independent set of multiple binding sites is the most common model for binding experiment evaluations. The analytical solution for the total heat is determined by (Freire et al., 1990):

$$Q = V Δ H [ [ L ] + \frac{1 + [ M ] nK - \sqrt{{( 1 + [ M ] nK - [ L ] K )}^{2} + 4 K [ L ]}}{2 K} ] \qquad ( 1 )$$

Where Q is the total heat, V is the cell volume, ΔH is the enthalpy, M is the macromolecule concentration (the binding partner in the cell), n is the binding stoichiometry, L is the ligand concentration (the binding partner in the syringe), and K is the association constant. Data were fit to this model using Origin version 5 (MicroCal, Inc.).

Surface Plasmon Resonance

The BiaCore-X surface plasmon resonance (SPR) device (BiaCore, Inc.) was utilized to measure the interaction between the PM4 complex (PM4-mAb-Pacific Blue) and bound human serum albumin. For these experiments a carboxymethyl dextran sensor chip (CM-5) was activated with 50 mM N-hydroxysuccinimide, 0.2 M N-ethyl-N′-(dimethylaminopropyl)-carbodiimide at a flow rate of 10 μL per minute for ten minutes. Anti human serum polyclonal antibodies, at a concentration of 100 ng/mL, was then coupled to the activated surface at a flow rate of 10 μL per minute for ten minutes. The final surface was inactivated by flowing 1 M ethanolamine-HCl at a rate of 10 μL per minute for five minutes over the sensor surface. Human serum albumin, at a concentration of 100 ng/mL, was then allowed to bind to the immobilized antibody (all flow rates at 10 μL/min.). The PM4 complex was flowed over the sensor surface at a rate of 30 μL per minute, and a concentration of 0.01% (v/v). The association and dissociation phases of the binding isotherms were smoothed by an automated FFT routine prior to modeling rate constants. Binding isotherms were evaluated by simultaneously fitting the forward (kₐ) and reverse (k_{d}) rate constants to:

d[mAb˜PM4]/dt=(kₐ[mAb] [PM4])−(k_{d}[mAb˜PM4]) (2)

(Karlsson and Falt, 1997) where [mAb], [PM4], and [mAb˜PM4] are the concentrations of the free monoclonal antibody, free PM4, and the complex respectively. The equilibrium affinity constant (K_{A}) is then defined as:

K_{A}=kₐ/k_{d} (3)

Equation 3 is properly expressed in terms of the SPR signal (Morton et al., 1995) as:

dR/dt=kₐCRₘₐₓ−(kₐC+k_{d})R (4)

where R is the SPR signal (in response units, RU) at time t, Rₘₐₓ is the maximum mAb binding capacity in RU, and C is the PM4 concentration. Kinetic analysis (O'Shannessy et al., 1993) was performed using Origin from Microcal, Inc.

### EXAMPLE 2

### Proteinoid Microsphere Immunoassay

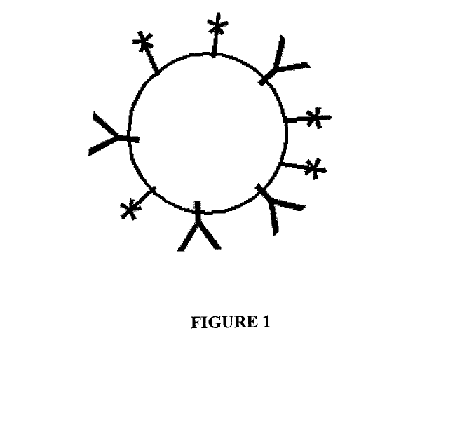

Proteinoid microspheres were derivatized with both an antibody and a fluorophore using simple carbodiiamide chemistry as described above. The free PM amines can be modified directly using the pre-formed succinimide ester of Pacific Blue. The free carboxylates on the PM can then be converted into a reactive succinimide ester and reacted with a thiol coupling reagent (PDEA). The resulting chemical species then undergoes a thiol exchange reaction with a thiolated antibody. A schematic diagram of the final PM4-fluorophore-antibody complex is depicted in FIG. 1. No attempt was made to optimize or titrate the number of fluorophore molecules or antibodies on the surface of PM4. It is therefore possible to increase the fluorescent signal from a PM complex by adding more Pacific Blue (or other fluorophore) molecules to the surface. Likewise, the optimal number of surface bound antibodies can be determined by titration. Even without any optimization, the system works well.

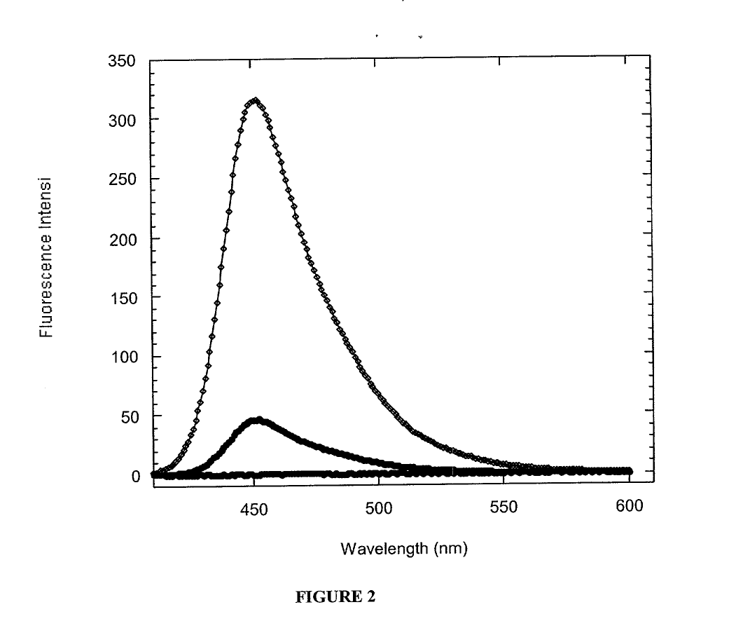

FIG. 2 shows the steady state fluorescence emission spectra for the PM4 complex relative to an equal concentration of Pacific Blue derivatized monoclonal antibody. Integration of the fluorescence emission intensity peaks (between 415 and 550 nm) reveals that there is a 7.3 fold increase in signal of the PM4 complex relative to the antibody value. This may indicate that seven fluorophores are coupled on average to the PM4 surface, although this number has not been directly determined.

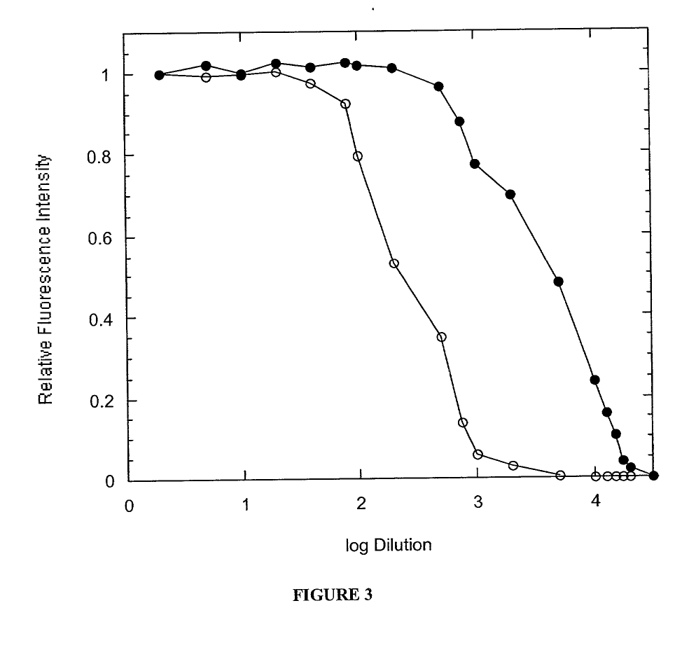

ELISA analysis indicates that the PM4 complex is 30 fold more sensitive than is a regular antibody. FIG. 3 shows the results from a typical ELISA reaction. The PM4 complex can be used at a 30 fold lower concentration compared to the labeled antibody preparation.

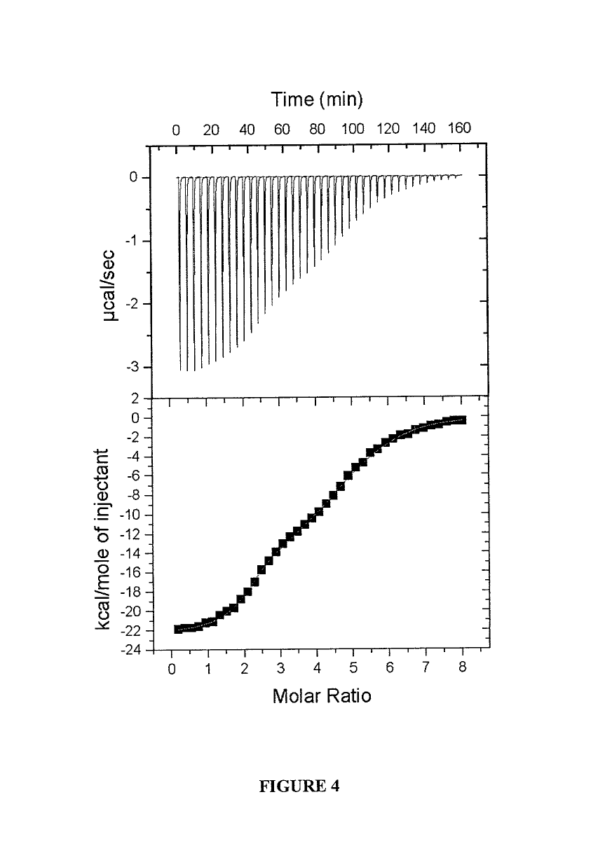

The PM4 complex can effectively bind to human serum albumin in solution. FIG. 4 shows the results of a binding reaction that was monitored by isothermal titration calorimetry. The binding isotherm shows that approximately four albumin molecules bind per PM4 complex. This is perhaps the best estimation of the number of functional antibodies (on average) per PM. Thermodynamic results indicate two classes of binding sites, as shown in the table below.

| | Site Type 1 | Site Type 2 |
|---|---|---|
| Stoichiometry: | 2.05 +/− 0.03 | 2.11 +/− 0.01 |
| ΔH (kcal/mol): | −2.24 × 10⁴ | −3.50 × 10⁴ |
| ΔS (cal mol⁻¹ K⁻¹) | −22.1 | −16.1 |
| Kₐ (M⁻¹): | 8.60 × 10⁶ | 3.6 × 10⁵ |

In both cases the binding is energetically favorable. The equilibrium binding affinity differences between the two site classes may arise from differences in antibody orientation on the PM surface or from molecular crowding of some of the antibodies.

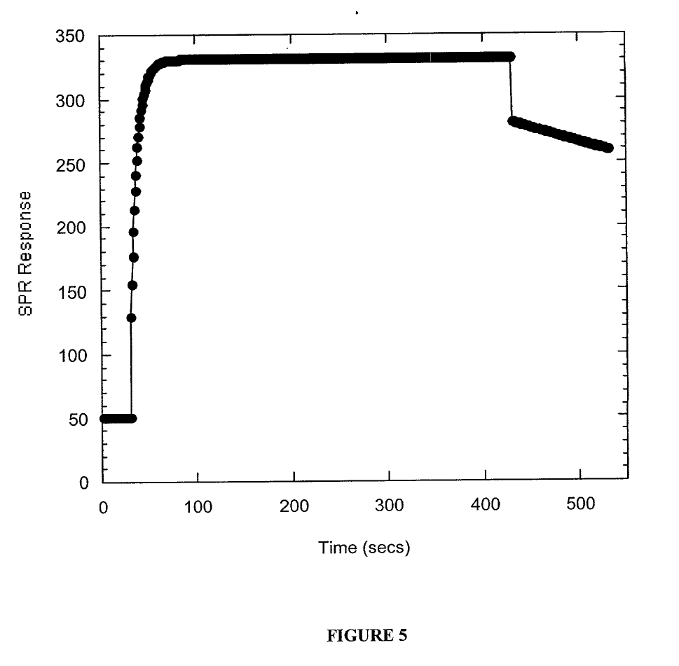

PM4 complex binding to human serum albumin was also studied kinetically via surface plasmon resonance (SPR). A CM-5 chip surface of polyclonal anti human serum albumin antibodies was prepared as described in Methods. This surface was reacted with human serum albumin (HSA) such that all of the chip-bound antibody was also bound to HSA. The PM4 complex was passed over this surface, and the binding isotherm is shown in FIG. 5. PM4 complex binds to the HSA/antibody surface with an affinity rate constant of 3.1×10⁵ M⁻¹s⁻¹.

The data obtained as described in Examples 1 and 2 indicate that stable PMs can be chemically modified and used to amplify the fluorescence intensity in an antibody based diagnostic device. A simple proteinoid microsphere that contained amino acids with amino or carboxyl sidechains (and a serine hydroxyl, which could be used to couple a third chemical type to the PM, although it was not used here) was formed. This PM was then reacted with the fluorophore Pacific Blue and an anti human serum albumin mAb. This complex was able to detect the presence of HSA in a surface plasmom resonance, in a isothermal titration calorimetric, and in an ELISA assay. The complex can be used at lower concentrations itself, or detect HSA at lower concentrations due to the amplification of the fluorescence signal. This amplification is a result of coupling multiple fluorophore molecules to the PM surface. In this instance there are approximately 7 fluorophores on the PM4 surface on average. Hence the signal for a single antibody-HSA binding event is magnified seven-fold. No attempt has been made to optimize the complex (either in the number of fluorophores or the number of antibodies bound). Such optimization will only result in increased performance of the material.

### REFERENCES

The following references, to the extent that they provide exemplary procedural or other details supplementary to those set forth herein, are specifically incorporated herein by reference.

Berger, G., 1984, [Hypotheses on the establishment of a genetic code and transfer of information from proteinoids to nucleic acids], C R Acad Sci III 299(9):333-8.

Bradford, M. M. (1976). A rapid and sensitive method for the quantitation of microgram quantities of protein utilizing the principle of dye binding. Anal. Biochem. 72, 248-254.

Brooke, S., and Fox, S. W., 1977, Compartmentalization in proteinoid microspheres, Biosystems 9(1):1-22.

Fox, S. W., and Harada, K., 1960, The thermal copolymerization of amino acids common to protein, J. Am. Chem. Soc. 82: 3745-51.

Fox, S. W., and Nakashima, T., 1974, Fractionation and characterization of an amidated thermal 1:1:1 proteinoid, Biochemica et Biophysica Acta 140: 155-167.

Fox, S. W., and Nakashima, T., 1980, The assembly and properties of protobiological structures: The beginnings of cellular peptide synthesis, Biosystems 12: 155-66.

Fox, S. W., Jungck, J. R., and Nakashima, T., 1974, From proteinoid microsphere to contemporary cell: formation of internucleotide and peptide bonds by proteinoid particles, Orig Life 5(1):227-37.

Fox, S. W., 1964, Thermal polymerization of amino acids and production of formed microparticles on lava, Nature 201: 336-37.

Fox, S. W., 1976, The evolutionary significance of phase-separated microsystems, Orig Life 7(1):49-68.

Fox, S. W., 1984, Self-sequencing of amino acids and origins of polyfunctional protocells, Orig Life 14(1-4):485-8.

Fox, S. W., 1991, Synthesis of life in the lab? Defining a protoliving system, Q Rev Biol 66(2):181-5.

Freire, E., van Osdol, W W., Mayorga, O L, and Sanchez-Ruiz, J M. (1990). Calorimetrically determined dynamics of complex unfolding transitions in proteins. Annu Rev Biophys Biophys Chem. 19, 159-88.

Green, N. S., Reisler, E., and Houk, K. N., 2001, Quantitative evaluation of the lengths of homobifunctional protein cross-inking reagents used as molecular probes, Prot. Sci., 10:1293-1304.

Harada, K., and Fox, S. W., 1958, The thermal condensation of glutamic acid and glycine to linear peptides, J. Am. Chem. Soc. 80: 2694-97.

Harada, K., and Fox, S. W., 1960, The thermal copolymerization of aspartic acid and glutamic acid, Arch. of Biochem. Biophys. 86: 274-80.

Hartmann, J., Brand, M. C., and Dose, K., 1981, Formation of specific amino acid sequences during thermal polymerization of amino acids, Biosystems 13: 141-47.

Hsu, L. L., Brooke, S., and Fox, S. W., 1971, Conjugation of proteinoid microspheres: a model of primordial communication, Curr Mod Biol 4(1):12-25.

Hsu, L. L., and Fox, S. W., 1976, Interactions between diverse proteinoids and microspheres in simulation of primordial evolution, Biosystems 8(2):89-101.

Ishima, Y., Przybylski, A. T., and Fox, S. W., 1981, Electrical membrane phenomena in spherules from proteinoid and lecithin, Biosystems 13(4):243-51.

Jungck, J. R., and Fox, S. W., 1973, Synthesis of oligonucleotides by proteinoid microspheres acting on ATP, Naturwissenschaften 60(9):425-7.

Kokufuta, E., Sakai, H., and Harada, K., 1983, Factors controlling the size of proteinoid microspheres, Biosystems 16(3-4):175-81.

Lakowicz, J. R. (1983). Principles of Fluorescence Spectroscopy, Chapter 10, Plenum Press, New York, London.

Luque-Romero, M. M., de Medina, L. S., and Blanco, J. M., 1986, Fractionation and amino acid composition of an aspartic acid-containing thermal proteinoid population, Biosystems 19(4):267-72.

Karlsson, R., and Falt, A. (1997). Experimental design for kinetic analysis of protein-protein interactions with surface plasmon resonance biosensors. J. Immunol. Meths. 200, 121-33.

Ma, X., Santiago, N., Chen, Y. S., Chaudhary, K., Milstein, S. J., and Baughman, R. A., 1994, Stability study of drug-loaded proteinoid microsphere formulations during freeze-drying, J Drug Target 2(1):9-21.

Madhan Kumar, A. B., and Panduranga Rao, K., 1998, Preparation and characterization of pH-sensitive proteinoid microspheres for the oral delivery of methotrexate, Biomaterials 19(7-9):725-32.

Masinovsky, Z., Lozovaya, G. I., Sivash, A. A., and Drasner, M., 1989, Porphyrin-proteinoid complexes as models of prebiotic photosensitizers, Biosystems 22(4):305-10.

Masinovsky, Z., 1995, [The origin and early development of biological catalysts], Cas Lek Cesk 134(19):607-10.

Matsuno, K., 1981, Self-sustaining multiplication and reproduction of Microsystems in protobiogenesis, Biosystems 14(2):163-70.

Matsuno, K., 198 lb, Material self-assembly as a physiochemical process, Biosystems 13: 237-241.

Matsuno, K., 1984, Electrical excitability of proteinoid microspheres composed of basic and acidic proteinoids, Biosystems 17(1):11-4.

McAlhaney, W. W., and Rohlfing, D. L., 1976, Formation of proteinoid microspheres under simulated prebiotic atmospheres and individual gases, Biosystems 8(2):45-50.

Morton, T. A., Myska, D. G., and Chaiken, I. M. (1995). Interpreting complex binding kinetics from optical biosensors: A comparison of analysis by linearization, the integrated rate equation, and numerical integration. Anal. Biochem. 227, 176-185.

Muller-Herold, U., and Nickel, G., 1994, The stability of proteinoid microspheres, Biosystems 33(3):215-20.

Nakashima, T., and Fox, S. W., 1980, Synthesis of peptides from amino acids and ATP with lysine-rich proteinoid, J Mol Evol 15(2):161-8.

Nakashima, T., and Fox, S. W., 1981, Formation of peptides from amino acids by single or multiple additions of ATP to suspensions of nucleoproteinoid microparticles, Biosystems 14(2):151-61.

O'Shannessy, D. J., Brigham-Burke, M., Soneson, K. K, Hensley, P., and Brooks, I. (1993). Determination of rate and equilibrium binding constants for macromolecular interactions using surface plasmon resonance: use of non linear least squares analysis methods. Anal. Biochem. 212, 457-468.

Phillips, R. D., and Melius, P., 1974, The thermal polymerization of amino acids, Int. J Peptide Protein Res. 6: 309-319.

Przybylski, A. T., Stratten, W. P., Syren, R. M., and Fox, S. W., 1982, Membrane, action, and oscillatory potentials in simulated protocells, Naturwissenschaften 69(12):561-3.

Przybylski, A. T., 1985, Excitable cell made of thermal proteinoids, Biosystems 17(4):281-8.

Rohlfing, D. L., 1975, Coacervate-like microspheres from lysine-rich proteinoid, Orig Life 6(1-2):203-9.

Ryan, J. W., and Fox, S. W., 1973, Activation of glycine by ATP, a divalent cation, and proteinoid microspheres, Curr Mod Biol 5(3):115-8.

Santiago, N., Milstein, S., Rivera, T., Garcia, E., Zaidi, T., Hong, H., and Bucher, D., 1993, Oral immunization of rats with proteinoid microspheres encapsulating influenza virus antigens, Pharm Res 10(8):1243-7.

Snyder, W. D., and Fox, S. W., 1975, A model for the origin of stable protocells in a primitive alkaline ocean, Biosystems 7(2):222-9.

Syren, R. M., Sanjur, A., and Fox, S. W., 1985, Proteinoid microspheres more stable in hot than in cold water, Biosystems 17(4):275-80.

The references and disclosures cited herein incorporated by reference in their entirety.

What is claimed:

1. A labeled proteinoid microsphere comprising a mixture of amino acids that are condensed and a label comprising a fluorophore, a chemiluminescent molecule, a radioisotope, a paramagnetic ion, a metal, or an enzyme.

2. The labeled proteinoid microsphere of claim 1 wherein the label is barium sulfate, iocetamic acid, iopanoic acid, ipodate calcium, diatrizoate sodium, diatrizoate meglumine, metrizamide, tyropanoate sodium, fluorine-18, carbon-11, iodine-123, technitium-99m, iodine-131, indium-111, fluorine, gadolinium, fluorescein, isothiocyalate, rhodamine, pacific blue, phycoerythrin, phycocyanin, allophycocyanin, ophthaldehyde, fluorescamine, luminal, isoluminal, luciferin, luciferase or aequorin.

3. The labeled proteinoid microsphere of claim 1 wherein the proteinoid microsphere is formed by thermal condensation of a mixture of amino acids in the presence of a crosslinking agent.

4. The labeled proteinoid microsphere of claim 3 wherein the crosslinking agent is carbodiimide, glutaraldehyde, N-(m-maleimidobenzoyloxy)-succinimide, a bifunctional sulfhydral reagent.

5. The labeled proteinoid microsphere of claim 1 that is used for signal amplification or diagnostic imaging.

6. The labeled proteinoid microsphere of claim 1 wherein the mixture of amino acids are thermally condensed.

7. A labeled proteinoid microsphere that is capable of binding to a specific target comprising a proteinoid microsphere linked to a label and a selective binding moiety that can bind to a specific target.

8. The labeled proteinoid microsphere of claim 6 wherein the label comprises a fluorophore, a chemiluminescent molecule, a radioisotope, a paramagnetic ion, a metal, or an enzyme.

9. The labeled proteinoid microsphere of claim 6 wherein the label is barium sulfate, iocetamic acid, iopanoic acid, ipodate calcium, diatrizoate sodium, diatrizoate meglumine, metrizamide, tyropanoate sodium, fluorine-18, carbon-11, iodine-123, technitium-99m, iodine-131, indium-111, fluorine, gadolinium, fluorescein, isothiocyalate, rhodamine, pacific blue, phycoerythrin, phycocyanin, allophycocyanin, ophthaldehyde, fluorescamine, luminal, isoluminal, luciferin, luciferase or aequorin.

10. The labeled proteinoid microsphere of claim 6 wherein the proteinoid microsphere is formed by thermal condensation of a mixture of amino acids in the presence of a crosslinking agent.

11. The labeled proteinoid microsphere of claim 9 wherein the crosslinking agent is carbodiimide, glutaraldehyde, N-(m-maleimidobenzoyloxy)-succinimide, a bifunctional sulfhydral reagent.

12. The labeled proteinoid microsphere of claim 6 wherein the selective binding moiety is an antibody, a ligand, a receptor, a peptide, a peptidyl analogue or a polypeptide.

13. The labeled proteinoid microsphere of claim 6 that is used in an immunoassay.

14. The labeled proteinoid microsphere of claim 12 wherein the immunoassay is a radioimmunoassay, an ELISA, an immunofluorescence assay or a sandwich assay.

15. The labeled proteinoid microsphere of claim 6 that is used for diagnostic imaging or signal amplification.

16. The labeled proteinoid microsphere of claim 6 wherein the signal amplification is at least about thirty-fold relative to an antibody preparation linked to the same label.

17. A labeled proteinoid microsphere that is capable of binding to a specific target comprising a proteinoid microsphere linked to a label and an antibody that can bind to a specific target.

18. The labeled proteinoid microsphere of claim 17 wherein the proteinoid microsphere comprises a thermally-condensed mixture of amino acids comprising an acidic amino acid and a basic amino acid.

19. The labeled proteinoid microsphere of claim 17 wherein the label comprises a fluorophore, a chemiluminescent molecule, a radioisotope, a paramagnetic ion, a metal, or an enzyme.

20. The labeled proteinoid microsphere of claim 17 wherein the acidic amino acid is aspartic acid or glutamic acid or a mixture of both.

21. The labeled proteinoid microsphere of claim 17 wherein the basic amino acid is arginine or lysine, or a mixture of both.

22. The labeled proteinoid microsphere of claim 17 wherein the mixture of amino acids further comprises cysteine.
